(19) United States
(12) Patent Application Publication
(10) Pub. No.: US 2026/0227406 A1
(43) Pub. Date: Aug. 6, 2026

(54) METHODS OF SUPERMERE DETECTION AND QUANTIFICATION

(71) Applicant: UNIVERSITY OF NOTRE DAME DU LAC, South Bend, IN (US)

(72) Inventors: Satyajyoti SENAPATI, South Bend, IN (US); Hsueh-Chia CHANG, South Bend, IN (US); Sonu KUMAR, South Bend, IN (US)

(21) Appl. No.: 19/151,444

(22) PCT Filed: Apr. 22, 2024
(86) PCT No.: PCT/US2024/025696

§ 371 Date: Jul. 28, 2025

Related U.S. Application Data
(60) Provisional application No. 63/463,064, filed on Apr. 30, 2023.

(51) Int. Cl. G01N 33/57535; G01N 27/414; G01N 33/543

(52) U.S. Cl. CPC G01N 33/57535; G01N 27/4145; G01N 33/54373

(57) ABSTRACT

Described herein is the novel discovery that supermeres comprise a high negative charge due to the presence of RNA-DNA hybrid molecules on their surface. The unique negative charge of supermeres, along with their specific protein content, can be used for detection and diagnostic applications. Also described herein are methods for detecting and quantifying supermeres in a sample that comprise the use of capture probes and charge-sensitive ion-selective sensors that are sensitive to the high negative charge of supermeres. Also described herein are methods for detecting cancer in a subject that comprise detecting and quantifying a concentration of one or more supermere-related cancer biomarkers.

### CROSS-REFERENCE TO RELATED APPLICATIONS

This application claims priority to U.S. Provisional Patent Application No. 63/463,064, filed on Apr. 30, 2023, which is incorporated by reference herein in its entirety.

### FEDERALLY SPONSORED RESEARCH

This invention was made with government support under grant number 1UH3CA241684-01 awarded by the National Institutes of Health. The government has certain rights in the invention.

### BACKGROUND

Liquid biopsy is an emerging non-invasive approach for the detection of circulating biomarkers in a sample of blood, saliva, urine, or other bodily fluid. Supermeres are the recently discovered functional extracellular nanoparticles secreted by cells that can contain a high amount of disease biomarkers and therapeutic targets. For example, supermeres are attractive cancer targets as there is evidence that tumor cells secrete more supermeres than any other biomarker or biomarker carriers (e.g., exosomes, lipoproteins, and ribonucleoproteins). However, a simple and rapid liquid biopsy assay for detecting and measuring levels of supermeres in plasma and other sample types is currently unavailable.

Currently, ELISA, western blot, PCR, next-generation sequencing (NGS), and mass spectrometry are commonly used to analyze the molecular cargo of supermeres after laborious and time-consuming isolation from other nanocarriers by ultracentrifugation. All these conventional techniques require purified supermeres to be lysed beforehand in order to extract their molecular cargo (e.g., proteins, nucleic acids, lipids, and N-glycosylation products) for analysis. ELISA techniques involving incubation, washing, and reading may take hours to complete. Western blotting may be less costly than ELISA, but requires multiple steps to conduct the analysis, and the entire processing time, including incubation, washing, and gel electrophoresis, is even longer than that of ELISA. By contrast, mass spectrometry provides the high-end capability for complex proteomic identification as it has both high sensitivity and high specificity. However, it requires intensive labor, sophisticated sample processing, and the equipment is typically too expensive for individual labs. For nucleic acid analysis, PCR and NGS are widely used due to their high sensitivity and reliability. In general, PCR is more cost-effective than NGS, whereas NGS is known for its high throughput and multiplexity. Nevertheless, both methods take long hours to complete, and a high level of expertise is required to decipher NGS data to obtain useful information.

What is needed are novel methods that provide a simple, rapid, robust, and ultrasensitive approach to detect and quantify supermeres from various types of samples. Such methods would be useful in a variety of commercial, research, and clinical applications, such as rapid disease screening for cancer.

### SUMMARY

One embodiment described herein is a method for detecting and quantifying supermeres in a sample, the method comprising: applying the sample to a charge-sensitive ion-selective sensor that is sensitive to a high negative charge of the supermeres; capturing the supermeres from the sample using one or more capture probes covalently attached to a surface of the charge-sensitive ion-selective sensor; and detecting and quantifying a concentration of the supermeres in the sample using the high negative charge of the supermeres. In one aspect, the capture probes comprise an anti-DDR1 antibody, an anti-HSPA13 antibody, an anti-ENO2 antibody, or combinations thereof that bind to the supermeres. In another aspect, the sample comprises cell culture media, whole blood, serum, plasma, urine, saliva, or cerebrospinal fluid (CSF). In another aspect, the sample comprises a plasma sample from a subject. In another aspect, the subject has cancer or is at risk of developing cancer. In another aspect, the cancer comprises colorectal cancer, stomach cancer, esophageal cancer, breast cancer, lung cancer, pancreatic cancer, skin cancer, brain cancer, neck cancer, ovarian cancer, cervical cancer, prostate cancer, kidney cancer, liver cancer, blood cancer, lymphoma, or combinations thereof. In another aspect, the cancer comprises colorectal cancer. In another aspect, the sample further comprises other non-supermere extracellular particles that are not captured, detected, or quantified by the method. In another aspect, the other non-supermere extracellular particles in the sample comprise extracellular vesicles (EVs), small extracellular vesicles (sEVs), exosomes, exomeres, ectosomes, microvesicles, lipoproteins, or combinations thereof. In another aspect, the high negative charge of the supermeres is due to the presence of RNA-DNA hybrids on a surface of the supermeres. In another aspect, the supermeres have a zeta potential equal to or greater than about −30 mV. In another aspect, the charge-sensitive ion-selective sensor comprises an anion exchange membrane sensor, wherein the capture probes are covalently attached to a surface of the anion exchange membrane sensor. In another aspect, detecting and quantifying the concentration of the supermeres in the sample comprises: removing physically adsorbed material from the surface of the anion exchange membrane sensor following capture of supermeres; applying an electric field to the anion exchange membrane sensor; recording a target current-voltage (I-V) signal on the anion exchange membrane sensor, wherein the sensor produces a target I-V signal current-voltage curve (CVC) with distinct underlimiting, limiting, and overlimiting regions; and comparing the target I-V signal CVC to a baseline I-V signal CVC, wherein when supermeres are bound to the capture probes attached to the surface of the anion exchange membrane sensor, the voltage responsible for the overlimiting region of the target I-V signal CVC is shifted as compared to an overlimiting region of the baseline I-V signal CVC, thereby confirming detection of supermeres in the sample, and wherein the concentration of the supermeres in the sample is directly correlated with a voltage shift value of the overlimiting region of the target I-V signal CVC. In another aspect, the charge-sensitive ion-selective sensor comprises an ion-sensitive field-effect transistor (ISFET) sensor, wherein the capture probes are covalently attached to a surface of the ISFET sensor. In another aspect, the method has a limit of detection of about 1×10⁵ to about 1×10⁷ supermeres/mL in the sample with 3 to 4 orders of dynamic range. In another aspect, the supermeres in the sample are detected and quantified in less than about 30 minutes.

Another embodiment described herein is a method of detecting cancer in a subject, the method comprising: capturing supermeres from a sample from the subject using one or more capture probes; and detecting and quantifying a concentration of one or more supermere-related cancer biomarkers in the sample from the subject. In one aspect, the capture probes comprise an anti-DDR1 antibody, an anti-HSPA13 antibody, an anti-ENO2 antibody, or combinations thereof that bind to the supermeres. In another aspect, the supermere-related cancer biomarkers comprise TGFB1, ENO1, DPEP1, GPC1, CEA, DDR1, HSPA13, ENO2, or combinations thereof. In another aspect, the sample further comprises other non-supermere extracellular particles. In another aspect, the other non-supermere extracellular particles in the sample comprise extracellular vesicles (EVs), small extracellular vesicles (sEVs), exosomes, exomeres, ectosomes, microvesicles, lipoproteins, or combinations thereof. In another aspect, the sample from the subject comprises whole blood, serum, plasma, urine, saliva, or cerebrospinal fluid (CSF). In another aspect, the sample comprises a plasma sample from the subject. In another aspect, the subject has cancer or is at risk of developing cancer. In another aspect, the cancer comprises colorectal cancer, stomach cancer, esophageal cancer, breast cancer, lung cancer, pancreatic cancer, skin cancer, brain cancer, neck cancer, ovarian cancer, cervical cancer, prostate cancer, kidney cancer, liver cancer, blood cancer, lymphoma, or combinations thereof.

This disclosure provides for other aspects and embodiments that will be apparent in light of the following detailed description and accompanying drawings.

### DESCRIPTION OF THE DRAWINGS

FIG. 1A-H show an overall summary of properties differentiating supermeres from sEVs and exomeres. FIG. 1A shows a schematic for an ultracentrifugation (UC) isolation procedure used for isolating sEV, exomere, and supermere fractions. FIG. 1B shows dynamic light scattering (DLS) results showing the different sizes of supermeres, exomeres, and sEVs. FIG. 1C shows that supermeres have a very negative zeta potential across multiple cell lines (magnitude>>R_{B}T/F), while exomeres and sEVs have weak zeta potentials in the same cell lines (magnitude<R_{B}T/F). FIG. 1D shows differential nuclease treatment on supermere zeta potential demonstrating that surface-exposed RNA-DNA duplexes likely give supermeres their very negative zeta potential, based on the significant RNase H effects that were observed. FIG. 1E shows the effect of RNase H treatment on the zeta potentials of sEVs, exomeres, and supermeres derived from multiple cell lines, confirming that the RNA-DNA duplexes are a unique feature on supermere surfaces that give supermeres their very negative zeta potential, while these charged duplexes are absent on the surfaces of exomeres and sEVs. FIG. 1F shows that supermeres have a similar detergent solubility profile as exomeres and sEVs. FIG. 1G (top) shows that HSPA13-positive supermeres and VCP-positive exomeres are positive markers for supermeres and exomeres, respectively (present in the majority of these particles). FIG. 1G (bottom) shows a heatmap of the expression of various proteins on sEVs, exomeres, and supermeres derived from multiple cell culture media, demonstrating that the proteins HSPA13, ENO2, and DDR1 are uniquely present on the majority of supermere particles compared to sEVs and exomeres. FIG. 1H shows that HSPA13 pulldown reduces the general protein concentration in solution containing supermeres, implying that supermeres are colocalized with the other protein markers.

FIG. 2A-G show an overall summary of the sensitivity and robustness of the disclosed ion exchange membrane sensor (IEMS) platform for isolation-free measurements. FIG. 2A shows an example schematic of the IEMS. FIG. 2B shows a characteristic current-voltage curve (CVC) for the IEMS, showing a shift in the overlimiting region upon binding of charged particles. FIG. 2C shows a calibration curve that allows voltage signal to be converted to supermere concentration. FIG. 2D shows that using the disclosed IEMS without isolation of cell media gives identical results for detecting supermere fractions as UC isolation methods that are followed by characterization with either IEMS or Surface Plasmon Resonance (SPR) (orthogonal measurements). FIG. 2E shows that RNase treatment reduced the signal of supermeres while detergent treatment broke the particles apart. Both treatments reduced the signal for IEMS, but RNase-treated supermeres produced a signal using SPR, validating that IEMS uses the zeta potential of supermeres for detection, while SPR uses changes in refractive index. FIG. 2F shows the measured concentrations of different protein markers from isolated fractions of supermeres, exomeres, sEVs, and non-vesicles (NVs). A signal was only produced from supermere fractions when a unique supermere protein marker was being measured, demonstrating the sensitivity of the disclosed IEMS for supermere-specific detection. FIG. 2G shows that when a shared protein marker (i.e., abundant in all fractions) such as CEA was measured, a signal was only produced in the supermere fraction.

FIG. 3A-C show that by measuring five different shared protein markers (TGFBI, ENO1, DPEP1, GPC1, and CEA) across all three fractions of supermeres, sEVs, and exomeres using UC+SPR, supermeres were found to outperform the other fractions in diagnosing colorectal cancer (CRC) patients. FIG. 3A shows a spider plot showing that supermeres had better areas under the curve (AUC) compared to exomeres and sEVs. FIG. 3B shows a spider plot showing that supermeres had better p-values compared to exomeres and sEVs. FIG. 3C shows the measured expression of TGFBI, ENO1, DPEP1, GPC1, and CEA protein markers in healthy and CRC patients for supermere, exomere, and sEV fractions using UC+SPR.

FIG. 4A-C show that supermere protein markers (HSPA13, DDR1, ENO2) outperformed sEV protein markers (CD63, CD81, EGFR) and exomere protein markers (FASN, VCP, POTEE) in diagnosing CRC patients, and that supermere markers showed a change in concentration after tumor resection surgery while sEV markers did not. FIG. 4A shows the measured expression of supermere protein markers (HSPA13, DDR1, ENO2), sEV protein markers (CD63, CD81, EGFR), and exomere protein markers (FASN, VCP, POTEE) in healthy and CRC patients using UC+SPR. FIG. 4B shows the measured protein expression for two independent patients (patient 1 (top) and patient 2 (bottom)) showing that the concentrations in the sEV subfraction were not significantly changed after tumor resection surgery. FIG. 4C shows the measured protein expression for two independent patients (patient 1 (top) and patient 2 (bottom)) showing that the concentrations in the supermere subfraction were changed (decreased) after tumor resection surgery.

FIG. 5A-B show that a 30-minute assay using the disclosed IEMS gave similar results as the 4-day long UC+SPR method for the protein marker CEA. FIG. 5A shows that a 30-minute assay using IEMS allowed for differentiation between healthy and CRC patients to be observed. FIG. 5B shows that a 4-day long UC+SPR method gave similar results as the IEMS method.

Before any embodiments of the disclosure are explained in detail, it is to be understood that the disclosure is not limited in its application to the details of construction and the arrangement of components set forth in the following description or illustrated in the following drawings. Also, it is to be understood that the phraseology and terminology used herein is for the purpose of description and should not be regarded as limiting.

### DETAILED DESCRIPTION

While the disclosure has been described in connection with certain embodiments, it is to be understood that the disclosure is not to be limited to the disclosed embodiments and is intended to cover various modifications and equivalent arrangements included within the scope of the appended claims, which scope is to be accorded the broadest interpretation to encompass all such modifications and equivalent structures as is permitted under the law.

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “gene,” “nucleic acid,” “nucleotide,” “polynucleotide,” “oligonucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein. Nucleic acids may be single stranded or double stranded or may contain portions of both double stranded and single stranded sequence. The nucleic acid may be DNA, both genomic and cDNA, RNA, or a hybrid, where the nucleic acid may contain combinations of deoxyribo-and ribo-nucleotides, and combinations of bases including uracil, adenine, thymine, cytosine, guanine, inosine, xanthine, hypoxanthine, isocytosine, and isoguanine. Nucleic acids may be obtained by chemical synthesis methods or by recombinant methods.

As used herein, terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not. As used herein, “comprising,” is an “open-ended” term that does not exclude additional, unrecited elements or method steps. As used herein, “consisting essentially of” limits the scope of a claim to the specified materials or steps and those that do not materially affect the basic and novel characteristics of the claimed invention. As used herein, “consisting of” excludes any element, step, or ingredient not specified in the claim.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points, or as described above in the definition of “about.”

As used herein, the terms “active ingredient” or “active pharmaceutical ingredient” refer to a pharmaceutical agent, active ingredient, compound, cell, or substance, compositions, or mixtures thereof, that provide a pharmacological, therapeutic, often beneficial, effect. In some embodiments, disclosed compositions may further comprise one or more pharmaceutically acceptable carriers or excipients. Example carriers may include, but are not limited to, liposomes, polymeric micelles, microspheres, microparticles, dendrimers, and/or nanoparticles.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifested.

As used herein, the term “sample” refers to any sample in which the presence and/or level of a target biomolecule (e.g., supermere) is to be detected or determined. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid (CSF), nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In some embodiments, the sample comprises a cell culture media sample. In other embodiments, the sample comprises a biological or bodily fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. In certain non-limiting exemplary embodiments, a sample comprises cell culture media, whole blood, serum, plasma, urine, saliva, or cerebrospinal fluid (CSF).

As used herein, the term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

As used herein, the term “cancer” refers to a disease characterized by the rapid and uncontrolled growth of aberrant cells. Cancer cells can spread locally or through the bloodstream and lymphatic system to other parts of the body (i.e., metastasize). Examples of various cancers include, but are not limited to, colorectal cancer, stomach cancer, esophageal cancer, breast cancer, lung cancer, pancreatic cancer, skin cancer, brain cancer, neck cancer, ovarian cancer, cervical cancer, prostate cancer, kidney cancer, liver cancer, blood cancer, lymphoma, or combinations thereof. In certain non-limiting exemplary embodiments, cancer comprises colorectal cancer.

As used herein, the terms “exosome,” “small extracellular vesicle,” and “sEV” are used interchangeably and refer to cell-derived and secreted membranous vesicles having a diameter of between about 30-200 nm, such as between about 40 and 140 nm, for example, a diameter of about 50 nm, 60 nm, 70 nm, 80 nm, 90 nm, 100 mm, 110 nm, 120 nm, 130 nm, or 150 nm. Exosomes may be isolated from any suitable biological sample from a mammal, including but not limited to, whole blood, serum, plasma, urine, saliva, breast milk, cerebrospinal fluid, amniotic fluid, ascitic fluid, bone marrow and cultured mammalian cells (e.g., immature dendritic cells (wild-type or immortalized), induced and non-induced pluripotent stem cells, fibroblasts, platelets, immune cells, reticulocytes, tumor cells, mesenchymal stem cells, satellite cells, hematopoietic stem cells, pancreatic stem cells, white and beige pre-adipocytes and the like). As one of skill in the art will appreciate, cultured cell samples will be in the cell-appropriate culture media (using exosome-free serum). Exosomes include specific surface markers not present in other vesicles, including surface markers such as tetraspanins, e.g., CD9, CD37, CD44, CD53, CD63, CD81, CD82 and CD151; targeting or adhesion markers such as integrins, ICAM-1, EpCAM and CD31; membrane fusion markers such as annexins, TSG101, ALIX; and other exosome transmembrane proteins such as Rab5b, HLA-G, HSP70, LAMP2 (lysosome-associated membrane protein) and LIMP (lysosomal integral membrane protein). Exosomes may also be obtained from a non-mammal or from cultured non-mammalian cells. As the molecular machinery involved in exosome biogenesis is believed to be evolutionarily conserved, exosomes from non-mammalian sources include surface markers which are isoforms of mammalian surface markers, such as isoforms of CD9 and CD63, which distinguish them from other cellular vesicles. The term “non-mammal” is meant to encompass, for example, exosomes from microorganisms such as bacteria, flies, worms, plants, fruit/vegetables (e.g., corn, pomegranate), and yeast.

As used herein, the term “exomere” refers to a secreted non-membranous extracellular nanoparticle having a diameter of equal to or less than about 100 nm, such as less than about 50 nm, often approximately 35 nm. Exomeres are highly enriched in metabolic enzymes and signature proteins involved in glycolysis and mTORCI signaling. In addition to proteins, nucleic acids and lipids are also selectively secreted in exomeres.

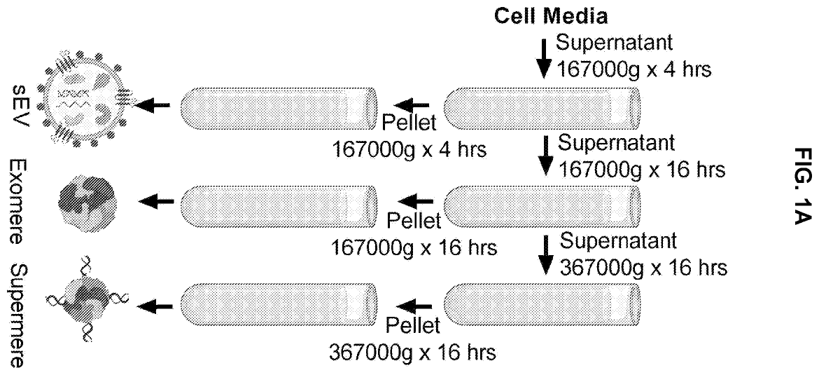

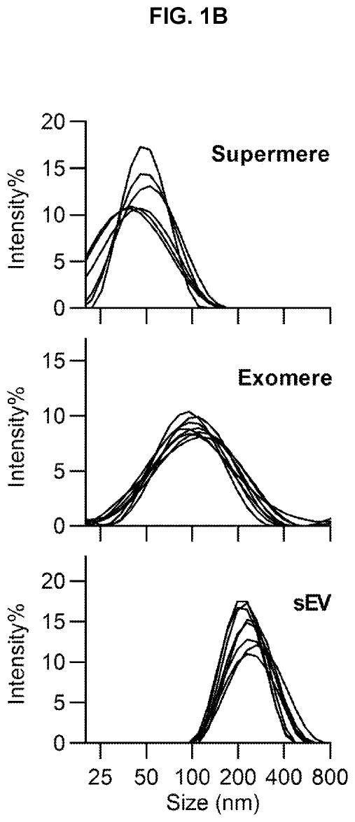

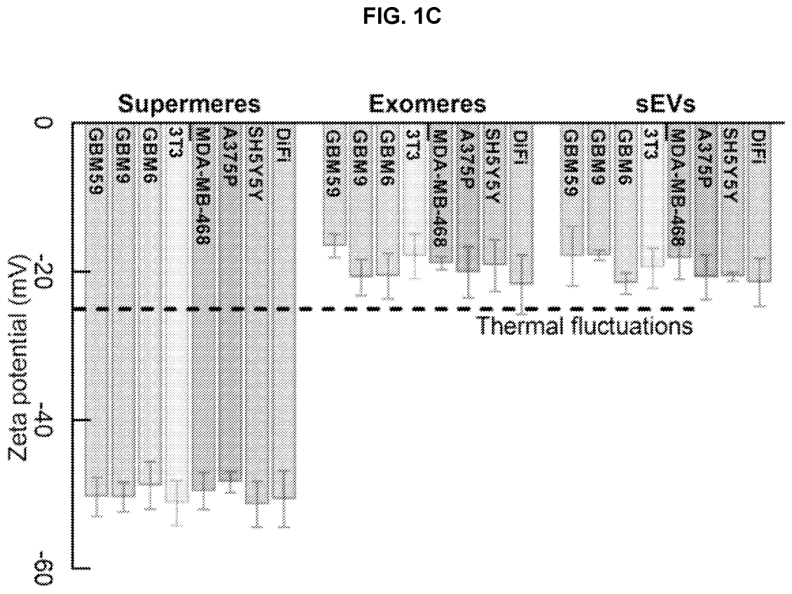

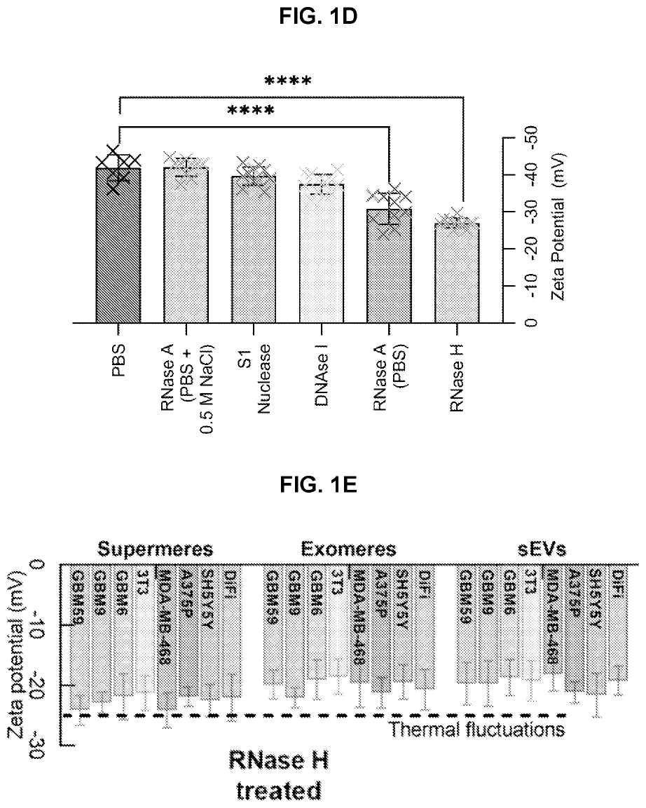

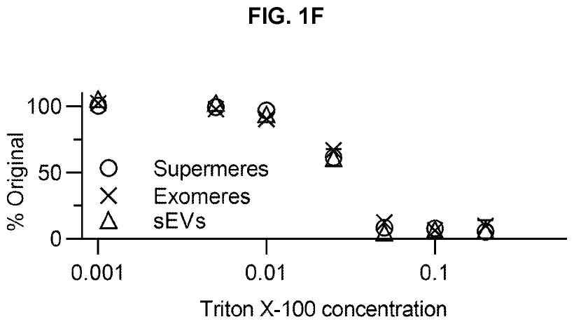

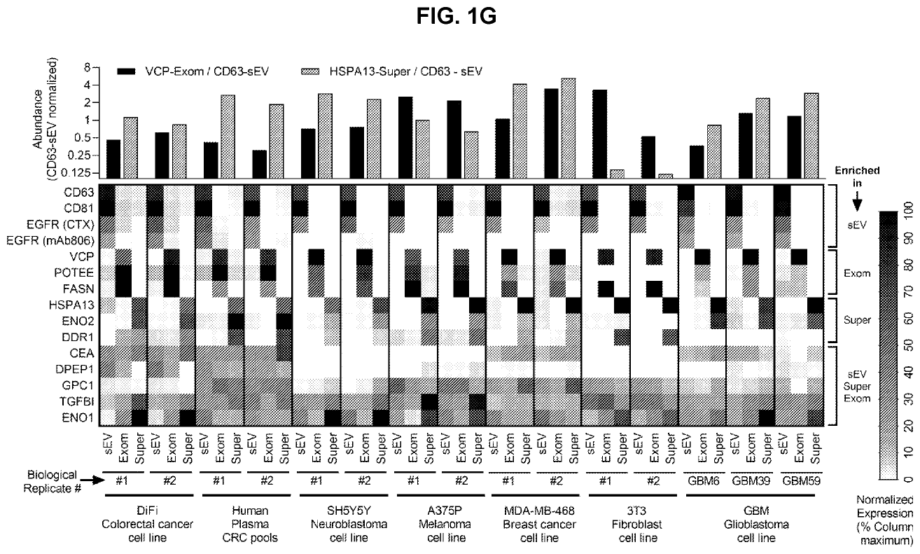

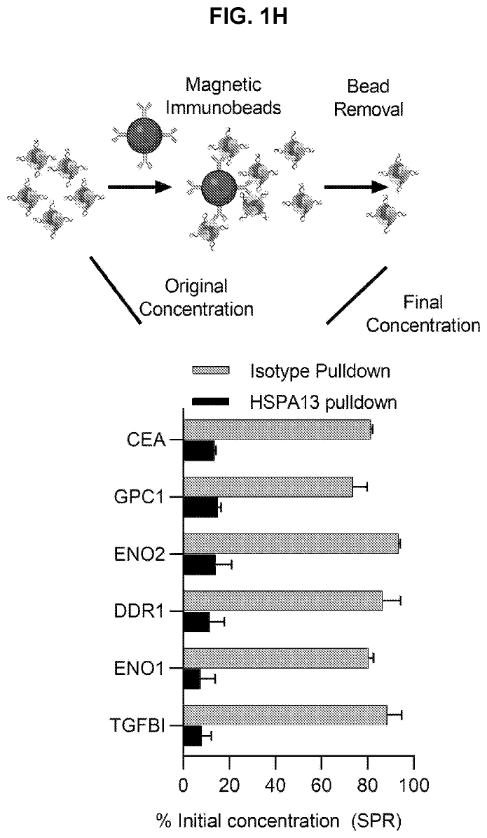

As used herein, the term “supermere” refers to a secreted non-membranous extracellular nanoparticle that can be isolated from the supernatant used to produce exomeres. For example, supermeres can be isolated by ultracentrifugation (UC) of the supernatant after the isolation of exomere particles, as shown in FIG. 1A. Supermeres are smaller than exomeres, ranging in diameter size from about 25 nm to about 50 nm, and are morphologically and molecularly distinct from exomeres. As disclosed herein, supermeres have a unique and high negative charge due to the presence of RNA-DNA hybrid molecules on the supermere surface. For example, supermeres may have a zeta potential of equal to or greater than about −30 mV, such as equal to or greater than about −40 mV, or even equal to or greater than about −50 mV, due to the presence of RNA-DNA hybrid molecules on the supermere surface. As used herein, the term “high negative charge of the supermeres” refers to a zeta potential of the supermeres that is equal to or greater than about −30 mV.

As used herein, the term “other non-supermere extracellular particles” refers to non-supermere extracellular particles that are not captured, detected, and/or quantified by the methods and sensors described herein. These other non-supermere extracellular particles can be spherical or non-spherical in shape. In some embodiments, the other non-supermere extracellular particles as described herein may have a size ranging from about 10 nm to about 500 nm, such as from about 30 nm to about 150 nm. In some embodiments, the other non-supermere extracellular particles as described herein may have a size that is greater than about 500 nm, such as from about 500 nm to about 1 μm. These other non-supermere extracellular particles can be charged or uncharged. In some embodiments, the other non-supermere extracellular particles as described herein may have a zeta potential of less than about −25 mV. For example, the other non-supermere extracellular particles as described herein may have a zeta potential of about −20 mV or less. In some embodiments, the other non-supermere extracellular particles as described herein may comprise extracellular vesicles (EVs), small extracellular vesicles (sEVs), exosomes, exomeres, ectosomes, microvesicles, lipoproteins, or combinations thereof. In certain non-limiting exemplary embodiments, the other non-supermere extracellular particles comprise exomeres and sEVs. In some embodiments, the disclosed methods and sensors are completely insensitive to interference from other non-supermere extracellular particles.

Disclosed herein is the novel discovery that supermeres comprise a high negative charge due to the presence of RNA-DNA hybrid molecules on their surface. This unique negative charge, along with the unique protein composition of supermeres, can be exploited for detection and diagnostic applications. Described herein are methods for detecting and quantifying supermeres in a sample that comprise the use of capture probes and charge-sensitive ion-selective sensors that are sensitive to the high negative charge of supermeres. Also described herein are methods for detecting cancer in a subject that comprise detecting and quantifying a concentration of one or more supermere-related cancer biomarkers.

The charge-sensitive ion-selective sensor-based platform described herein provides a simple, rapid, robust, and ultrasensitive method for detecting and measuring supermeres in samples in less than 30 minutes with a limit of detection of about 1×10⁵ to about 1×10⁷ supermeres/mL in the sample with 3 to 4 orders of dynamic range. Importantly, due to the newly discovered high negative charge of supermeres, the present disclosure demonstrates that the charge-sensitive ion-selective sensors, as described herein, can be insensitive to interference from non-supermere agents. The disclosed charge-sensitive ion-selective sensor-based platform was tested to detect supermeres in cell media and plasma samples and can be easily extended to other complex bodily fluids. As disclosed herein, supermere concentrations were found to be significantly higher in plasma samples of colorectal cancer (CRC) patients compared to healthy controls. Accordingly, the disclosed methods provide a novel approach for detecting and measuring supermeres for rapid disease screening.

The charge-sensitive ion-selective sensors disclosed herein can directly detect supermeres in the presence of other non-supermere extracellular particles, as they are insensitive to non-charged or weakly charged particles. Thus, the charge-sensitive ion-selective sensors do not produce a signal from free-floating proteins or other extracellular particles such as EVs and exomeres, even though the surface of the sensors may capture these other particles from samples. In some embodiments, the charge-sensitive ion-selective sensor may comprise an anion exchange membrane (AEM) sensor. In other embodiments, the charge-sensitive ion-selective sensor may comprise an ion-sensitive field-effect transistor (ISFET) sensor. An AEM-based sensing platform for analyzing other types of biomolecules, such as nucleic acids, has previously been described in U.S. Pat. Nos. 10,247,720, 10,557,820, and 11,016,079, each of which is incorporated by reference in its entirety into the specification.

Some particular advantages of the disclosed charge-sensitive ion-selective sensor technology over the conventional methods for supermere detection include: high sensitivity (low limit of detection (LoD) of about 1×10⁵ to about 1×10⁷ supermere particles/mL with 3 to 4 orders of dynamic range); rapid assay times (<30 min); small sample size requirement (only about 40-50 microliters for a single measurement); low cost (no particularly expensive facilities are required for chip/sensor fabrication or to run an assay); extension of the method to various bodily fluids; and the sensor technology can be easily integrated into a miniaturized device for point of care (POC) analysis of supermeres (e.g., on a millifluidic chip or similar device).

The disclosed charge-sensitive ion-selective sensors are capable of detecting and quantifying supermeres. The sensors are capable of being a standalone unit, but can also be used as part of an integrated chip, which can improve the sensitivity and the specificity of the sensors. These integrated chips are described in U.S. Pat. No. 11,016,079, which is incorporated by reference in its entirety into the specification. The sensors measure supermeres that hybridize to capture probes covalently attached to a surface of the sensor. In some embodiments, the supermeres may be transported and localized near the capture probes through pipette, or through other units in an integrated chip design, including a pre-treatment unit and a pre-concentration unit. After supermeres are captured and hybridized to capture probes, a period of hybridization is allowed, and the probes are washed to remove any non-specific or non-targeted molecules. Supermeres are then detected and quantified. Because supermeres are negatively charged, a positively charged membrane (e.g., AEM) can be used to detect the supermeres. The negative charge of the supermeres can be used to manipulate them, transport them, or to pre-concentrate them. At the same time, this negative charge can be thought of as an intrinsic tag used to detect and quantify the supermeres.

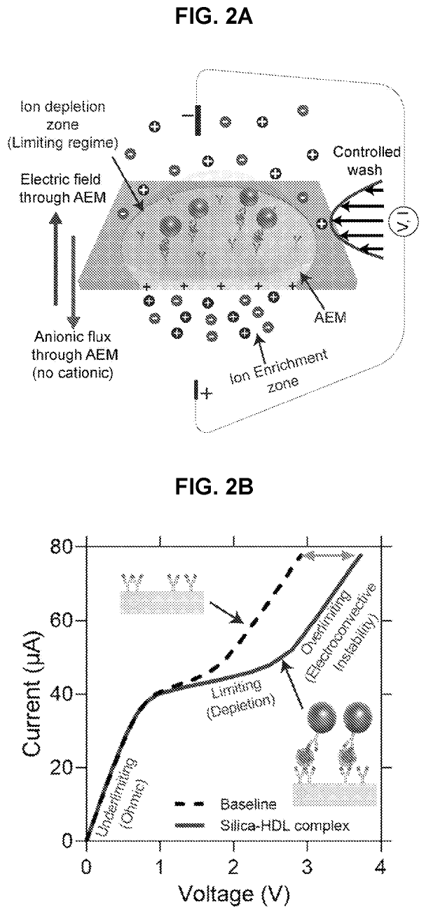

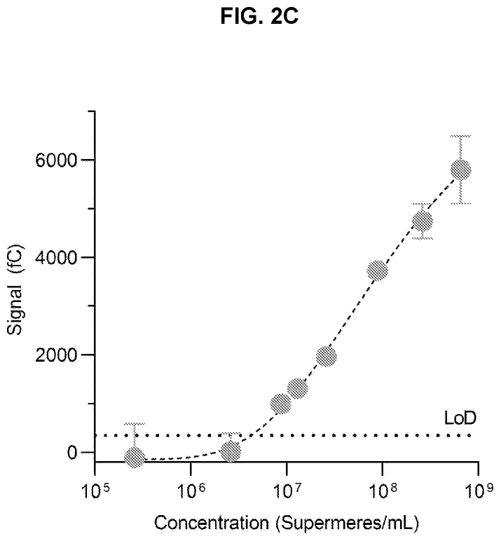

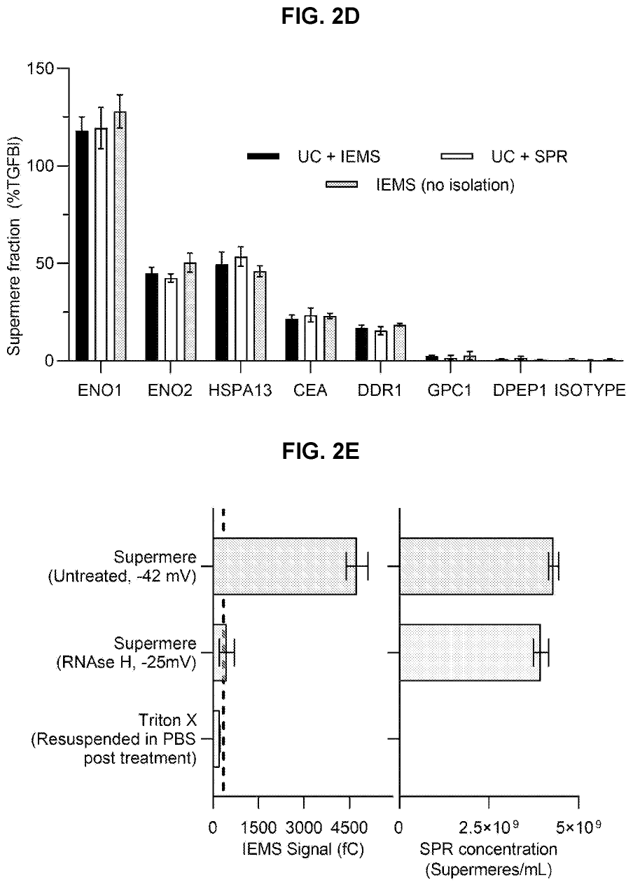

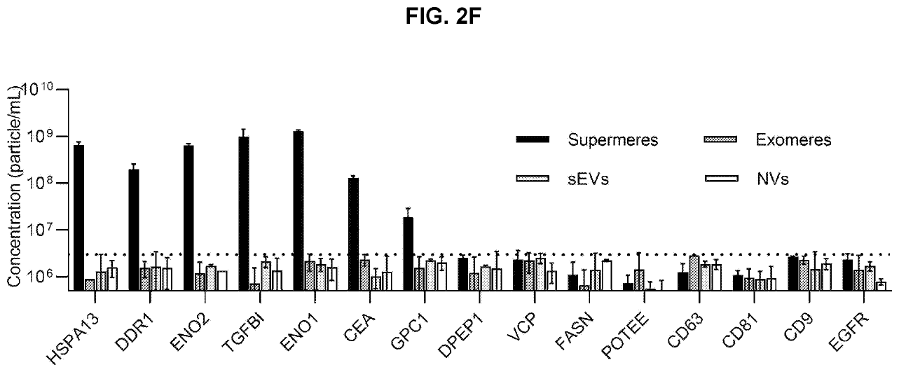

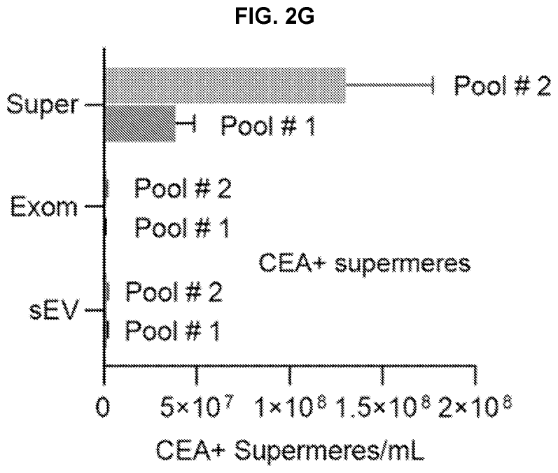

When the negatively charged supermere molecules are captured and fixed on the surface of the membrane sensors, such that the negatively charged supermere molecules are sitting on the positively charged membrane (e.g., AEM), the anions that would normally go through the membrane feel some repulsion. As a result, there is a measurable change that is detected with a current-voltage curve (CVC). In essence, the supermere biomolecules are drawn towards the membrane because the membrane is positively charged and the supermeres are negatively charged. The supermeres are adsorbed onto the surface of the membrane facilitated by the electrostatic interaction. The result is that one side of the membrane has an increase in ion concentration and the other side has a decrease in ion concentration. When measuring the CVC of this membrane, the result is a non-linear characteristic CVC having three main regions, underlimiting, limiting, and overlimiting, as shown in FIG. 2B. The limiting region is associated with the formation of the depletion zone of the membrane. The overlimiting region is associated with the mechanism that destroys this depletion. If an anion exchange membrane is combined with either a cation exchange membrane or with charged biomolecules, the curve shifts. The degree of shift in the curve is dependent upon the quantity and size of the biomolecules. Thus, the quantitation and characterization of supermere biomolecules is possible through the determination and characterization of the CVC. For characterization and quantitation of supermeres, the change in the voltage between two curves is measured. Prior to a current being applied, negatively charged supermere molecules near the membrane will begin to adsorb on the surface of the membrane. The anions near the membrane will move into the membrane as well. After the current is applied, more and more negatively charged supermere molecules move towards the membrane.

Initially as the voltage rises the negative ions move through the membrane creating a depletion zone. There are two phenomena that occur that cause the CVC to change from the underlimiting region to the limiting region to the overlimiting region. The first is microvortices and the second is water splitting. Both occur near the interface of the membrane. At the underlimiting region of the curve, anions move through the membrane and as these deplete the voltage increases across the membrane. The concentration zone is where the anions concentrate after moving through the membrane. There is a depletion, which means there is a small number of ions that carry the current, which is associated with a high voltage drop. At some point, the concentration of the ions in the depletion zone will be close to zero. At this point, more current cannot pass through the membrane. This is the beginning of the flattening of the CVC or the beginning of the limiting region of the CVC. At this point, there is a very high field which triggers motion in the electrolyte or a microvortice. When this happens, the depletion zone is destroyed. The mixing brings new ions that are further away from the membrane to the membrane and then the CVC begins to rise in a linear direction again, and this is the overlimiting region.

When supermere biomolecules adsorb to the membrane, the flux of the ions is changed. At the same time, the supermere biomolecules suppress the vortices. In the place of vortices is water splitting, as shown by the equation: H₂O→H⁺→OH⁻. A small layer exists between the membrane and the supermeres adsorption. The water molecules near the depletion zone are subjected to a high electrical force. This generates new ions as the water splits into positively charged hydrogen molecules and negatively charged hydroxide molecules. The result is an electrochemical reaction that separates the water molecules. The OH⁻ molecules travel in the direction of the membrane whereas the H⁺ molecules move in the opposite direction.

The permeability, or the size of the molecules that can travel through the membrane is about 200 g/mole in terms of molecular weight. Anything larger will not go through the membrane. Supermeres and other non-supermere extracellular particles are therefore too large to pass through the membrane.

The rate of water splitting depends on the concentration of the supermere molecules. If there is a large concentration, then the measured change will be more gradual than with lower concentrations. Therefore, the disclosed sensors have the ability to quantify the concentration of supermeres in a sample. The shift of the CVC from the baseline measurement, or the measurement without any captured supermeres, to the CVC with captured supermeres can determine both whether supermeres are present and the amount (i.e., concentration) of supermeres present.

When the concentration of supermere molecules is low in a sample, the vortices are delayed slightly and minimal water splitting occurs. When the concentration of supermere molecules is higher, there are fewer vortices and more water splitting that occurs. As a result, different CVC curves are measured and generated for different concentrations of supermeres. Thus, the further the shift of a CVC to the right from baseline, the higher the concentration of supermeres in a sample. In some embodiments described herein, detecting and quantifying a concentration of supermeres in a sample may comprise: removing physically adsorbed material from the surface of an anion exchange membrane sensor following capture of supermeres; applying an electric field to the anion exchange membrane sensor; recording a target current-voltage (I-V) signal on the anion exchange membrane sensor, wherein the sensor produces a target I-V signal current-voltage curve (CVC) with distinct underlimiting, limiting, and overlimiting regions; and comparing the target I-V signal CVC to a baseline I-V signal CVC, wherein when supermeres are bound to the capture probes attached to the surface of the anion exchange membrane sensor, the voltage responsible for the overlimiting region of the target I-V signal CVC is shifted as compared to an overlimiting region of the baseline I-V signal CVC, thereby confirming detection of supermeres in the sample, and wherein the concentration of the supermeres in the sample is directly correlated with a voltage shift value of the overlimiting region of the target I-V signal CVC.

The sensors disclosed herein allow for supermere hybridization and measuring the hybridization using CVC shifts from baseline. Because the ion exchange membranes cause vortices, these can be used to mix the supermere molecules near the sensor to accelerate hybridization. In addition, a field that is created with these membranes allows a system to move, separate, concentrate, and hold molecules in a desired location within a system. No mechanical force is necessary to complete all these tasks automatically.

Furthermore, reducing the size of the disclosed sensors means that fewer supermere molecules are required to hybridize to observe a shift in the CVC. Thus, with a smaller sensor, there may be a greater ability to detect supermeres in smaller concentrations. For example, in one non-limiting exemplary embodiment, the disclosed sensors and methods have a limit of detection (LoD) of about 1×10⁵ to about 1×10⁷ supermeres/mL in a sample with 3 to 4 orders of dynamic range.

One embodiment described herein is a method for detecting and quantifying supermeres in a sample, the method comprising: applying the sample to a charge-sensitive ion-selective sensor that is sensitive to a high negative charge of the supermeres; capturing the supermeres from the sample using one or more capture probes covalently attached to a surface of the charge-sensitive ion-selective sensor; and detecting and quantifying a concentration of the supermeres in the sample using the high negative charge of the supermeres.

In one aspect, the capture probes comprise an anti-DDR1 antibody, an anti-HSPA13 antibody, an anti-ENO2 antibody, or combinations thereof that bind to the supermeres.

In another aspect, the sample comprises cell culture media, whole blood, serum, plasma, urine, saliva, or cerebrospinal fluid (CSF).

In another aspect, the sample comprises a plasma sample from a subject.

In another aspect, the subject has cancer or is at risk of developing cancer.

In another aspect, the cancer comprises colorectal cancer, stomach cancer, esophageal cancer, breast cancer, lung cancer, pancreatic cancer, skin cancer, brain cancer, neck cancer, ovarian cancer, cervical cancer, prostate cancer, kidney cancer, liver cancer, blood cancer, lymphoma, or combinations thereof.

In another aspect, the cancer comprises colorectal cancer.

In another aspect, the sample further comprises other non-supermere extracellular particles that are not captured, detected, or quantified by the method.

In another aspect, the other non-supermere extracellular particles in the sample comprise extracellular vesicles (EVs), small extracellular vesicles (sEVs), exosomes, exomeres, ectosomes, microvesicles, lipoproteins, or combinations thereof.

In another aspect, the high negative charge of the supermeres is due to the presence of RNA-DNA hybrids on a surface of the supermeres.

In another aspect, the supermeres have a zeta potential equal to or greater than about −30 mV.

In another aspect, the charge-sensitive ion-selective sensor comprises an anion exchange membrane sensor, wherein the capture probes are covalently attached to a surface of the anion exchange membrane sensor.

In another aspect, detecting and quantifying the concentration of the supermeres in the sample comprises: removing physically adsorbed material from the surface of the anion exchange membrane sensor following capture of supermeres; applying an electric field to the anion exchange membrane sensor; recording a target current-voltage (I-V) signal on the anion exchange membrane sensor, wherein the sensor produces a target I-V signal current-voltage curve (CVC) with distinct underlimiting, limiting, and overlimiting regions; and comparing the target I-V signal CVC to a baseline I-V signal CVC, wherein when supermeres are bound to the capture probes attached to the surface of the anion exchange membrane sensor, the voltage responsible for the overlimiting region of the target I-V signal CVC is shifted as compared to an overlimiting region of the baseline I-V signal CVC, thereby confirming detection of supermeres in the sample, and wherein the concentration of the supermeres in the sample is directly correlated with a voltage shift value of the overlimiting region of the target I-V signal CVC.

In another aspect, the charge-sensitive ion-selective sensor comprises an ion-sensitive field-effect transistor (ISFET) sensor, wherein the capture probes are covalently attached to a surface of the ISFET sensor.

In another aspect, the method has a limit of detection of about 1×10⁵ to about 1×10⁷ supermeres/mL in the sample with 3 to 4 orders of dynamic range.

In another aspect, the supermeres in the sample are detected and quantified in less than about 30 minutes.

Another embodiment described herein is a method of detecting cancer in a subject, the method comprising: capturing supermeres from a sample from the subject using one or more capture probes; and detecting and quantifying a concentration of one or more supermere-related cancer biomarkers in the sample from the subject.

In one aspect, the capture probes comprise an anti-DDR1 antibody, an anti-HSPA13 antibody, an anti-ENO2 antibody, or combinations thereof that bind to the supermeres.

In another aspect, the supermere-related cancer biomarkers comprise TGFB1, ENO1, DPEP1, GPC1, CEA, DDR1, HSPA13, ENO2, or combinations thereof.

In another aspect, the sample further comprises other non-supermere extracellular particles.

In another aspect, the other non-supermere extracellular particles in the sample comprise extracellular vesicles (EVs), small extracellular vesicles (sEVs), exosomes, exomeres, ectosomes, microvesicles, lipoproteins, or combinations thereof.

In another aspect, the sample from the subject comprises whole blood, serum, plasma, urine, saliva, or cerebrospinal fluid (CSF).

In another aspect, the sample comprises a plasma sample from the subject.

In another aspect, the subject has cancer or is at risk of developing cancer.

In another aspect, the cancer comprises colorectal cancer, stomach cancer, esophageal cancer, breast cancer, lung cancer, pancreatic cancer, skin cancer, brain cancer, neck cancer, ovarian cancer, cervical cancer, prostate cancer, kidney cancer, liver cancer, blood cancer, lymphoma, or combinations thereof.

It will be apparent to one of ordinary skill in the relevant art that suitable modifications and adaptations to the compositions, formulations, methods, systems, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of any compositions, formulations, methods, systems, and processes described herein include all actual or potential combinations of embodiments, aspects, options, examples, and preferences herein described. Any exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A method for detecting and quantifying supermeres in a sample, the method comprising:
   applying the sample to a charge-sensitive ion-selective sensor that is sensitive to a high negative charge of the supermeres;
   capturing the supermeres from the sample using one or more capture probes covalently attached to a surface of the charge-sensitive ion-selective sensor; and
   detecting and quantifying a concentration of the supermeres in the sample using the high negative charge of the supermeres.
Clause 2. The method of clause 1, wherein the capture probes comprise an anti-DDR1 antibody, an anti-HSPA13 antibody, an anti-ENO2 antibody, or combinations thereof that bind to the supermeres.
Clause 3. The method of clause 1 or 2, wherein the sample comprises cell culture media, whole blood, serum, plasma, urine, saliva, or cerebrospinal fluid (CSF).
Clause 4. The method of any one of clauses 1-3, wherein the sample comprises a plasma sample from a subject.
Clause 5. The method of any one of clauses 1-4, wherein the subject has cancer or is at risk of developing cancer.
Clause 6. The method of any one of clauses 1-5, wherein the cancer comprises colorectal cancer, stomach cancer, esophageal cancer, breast cancer, lung cancer, pancreatic cancer, skin cancer, brain cancer, neck cancer, ovarian cancer, cervical cancer, prostate cancer, kidney cancer, liver cancer, blood cancer, lymphoma, or combinations thereof.
Clause 7. The method of any one of clauses 1-6, wherein the cancer comprises colorectal cancer.
Clause 8. The method of any one of clauses 1-7, wherein the sample further comprises other non-supermere extracellular particles that are not captured, detected, or quantified by the method.
Clause 9. The method of any one of clauses 1-8, wherein the other non-supermere extracellular particles in the sample comprise extracellular vesicles (EVs), small extracellular vesicles (sEVs), exosomes, exomeres, ectosomes, microvesicles, lipoproteins, or combinations thereof.
Clause 10. The method of any one of clauses 1-9, wherein the high negative charge of the supermeres is due to the presence of RNA-DNA hybrids on a surface of the supermeres.
Clause 11. The method of any one of clauses 1-10, wherein the supermeres have a zeta potential equal to or greater than about −30 mV.
Clause 12. The method of any one of clauses 1-11, wherein the charge-sensitive ion-selective sensor comprises an anion exchange membrane sensor, and wherein the capture probes are covalently attached to a surface of the anion exchange membrane sensor.
Clause 13. The method of any one of clauses 1-12, wherein detecting and quantifying the concentration of the supermeres in the sample comprises:
   removing physically adsorbed material from the surface of the anion exchange membrane sensor following capture of supermeres;
   applying an electric field to the anion exchange membrane sensor;
   recording a target current-voltage (I-V) signal on the anion exchange membrane sensor, wherein the sensor produces a target I-V signal current-voltage curve (CVC) with distinct underlimiting, limiting, and overlimiting regions; and
   comparing the target I-V signal CVC to a baseline I-V signal CVC, wherein when supermeres are bound to the capture probes attached to the surface of the anion exchange membrane sensor, the voltage responsible for the overlimiting region of the target I-V signal CVC is shifted as compared to an overlimiting region of the baseline I-V signal CVC, thereby confirming detection of supermeres in the sample, and wherein the concentration of the supermeres in the sample is directly correlated with a voltage shift value of the overlimiting region of the target I-V signal CVC.
Clause 14. The method of any one of clauses 1-13, wherein the charge-sensitive ion-selective sensor comprises an ion-sensitive field-effect transistor (ISFET) sensor, and wherein the capture probes are covalently attached to a surface of the ISFET sensor.
Clause 15. The method of any one of clauses 1-14, wherein the method has a limit of detection of about 1×10⁵ to about 1×10⁷ supermeres/mL in the sample with 3 to 4 orders of dynamic range.
Clause 16. The method of any one of clauses 1-15, wherein the supermeres in the sample are detected and quantified in less than about 30 minutes.
Clause 17. A method of detecting cancer in a subject, the method comprising:
   capturing supermeres from a sample from the subject using one or more capture probes; and
   detecting and quantifying a concentration of one or more supermere-related cancer biomarkers in the sample from the subject.
Clause 18. The method of clause 17, wherein the capture probes comprise an anti-DDR1 antibody, an anti-HSPA13 antibody, an anti-ENO2 antibody, or combinations thereof that bind to the supermeres.
Clause 19. The method of clause 17 or 18, wherein the supermere-related cancer biomarkers comprise TGFB1, ENO1, DPEP1, GPC1, CEA, DDR1, HSPA13, ENO2, or combinations thereof.
Clause 20. The method of any one of clauses 17-19, wherein the sample further comprises other non-supermere extracellular particles.
Clause 21. The method of any one of clauses 17-20, wherein the other non-supermere extracellular particles in the sample comprise extracellular vesicles (EVs), small extracellular vesicles (sEVs), exosomes, exomeres, ectosomes, microvesicles, lipoproteins, or combinations thereof.
Clause 22. The method of any one of clauses 17-21, wherein the sample from the subject comprises whole blood, serum, plasma, urine, saliva, or cerebrospinal fluid (CSF).
Clause 23. The method of any one of clauses 17-22, wherein the sample comprises a plasma sample from the subject.
Clause 24. The method of any one of clauses 17-23, wherein the subject has cancer or is at risk of developing cancer.
Clause 25. The method of any one of clauses 17-24, wherein the cancer comprises colorectal cancer, stomach cancer, esophageal cancer, breast cancer, lung cancer, pancreatic cancer, skin cancer, brain cancer, neck cancer, ovarian cancer, cervical cancer, prostate cancer, kidney cancer, liver cancer, blood cancer, lymphoma, or combinations thereof.

### EXAMPLES

### Isolation-Free Detection of Supermeres Using an Ion Exchange Membrane Sensor (IEMS) Platform

The following experimental examples examined the amembraneous extracellular nanoparticles, supermeres, using two orthogonal characterization methods—conventional Surface Plasmon Resonance (SPR) and an lon Exchange Membrane Sensor (IEMS) biosensing method. Supermeres are devoid of a membrane and are marked by a diverse and rich composition of proteins and nucleic acids implicated in the pathogenesis of various diseases. Unlike typical characterization methods such as SPR that first require supermere isolation using ultracentrifugation (UC) or chromatography methods over multiple days, the disclosed IEMS approach can detect supermeres directly from small volumes (e.g., ~100 uL) of complex biofluid such as biological media in significantly shorter times (e.g., <60 minutes), and has a limit of detection (LoD) of about 10⁵-10⁷ supermeres/mL. This was achieved by exploiting the newly discovered negative zeta potential of supermeres (>R_{B}T/F) due to RNA-DNA hybrids present on their surface that are absent on sEVs and exomeres, and a larger surface area than freely suspending nucleic acid species, giving the supermeres a very high effective charge when present in the debye layer.

Using the disclosed methods, supermeres were shown to have differential solubility under detergents, and also have a smaller size and a set of unique proteins that differ from sEVs and exomeres, while also sharing a set of proteins with these other nanoparticles. Specifically, HSPA13, DDR1, and ENO2 (NSE) were found to be present uniquely on supermeres, while VCP, FASN, and POTEE were found to be present uniquely on exomeres, and CD9, CD81, CD63, and EGFR were present uniquely on sEVs. Another group of proteins that was studied was found to be shared across the different supermere, exomere, and sEV fractions, including CEA, TGFBI, ENO1, DPEP1, and GPC1. These unique and shared sets of proteins on the different nanoparticles were used to determine the diagnostic potential of the different proteins and nanoparticles in diagnosing colorectal cancer.

The diagnostic studies found that, among the set of proteins shared across the three particle fractions, the proteins localized on supermeres gave significantly better results than the same proteins from the exomere and sEV fractions. Additionally, the proteins that were found to be more specific to supermeres (HSPA13, DDR1, and ENO2) outperformed the proteins found to be more specific in exomeres (VCP, POTEE, and FASN) and sEVs (CD63, CD81, CD9, and EGFR) in diagnosing colorectal cancer. Furthermore, patients who had recently undergone successful resection surgeries showed markedly reduced levels of supermeres using all the major proteins that were studied. Overall, these studies describe a novel platform for characterizing supermeres, and further demonstrate that supermeres may emerge as key biomolecules in diagnostic applications, similar to sEVs.

### Example 1

### Supermeres Differ in Size, Zeta Potential, and Protein Composition Compared to Exomeres and sEVs

Supermeres, exomeres, and sEVs were isolated using ultracentrifugation (UC) from several different cell media, including a control fibroblast cell media (FIG. 1A). Dynamic light scattering (DLS) was then performed to measure the size distribution of the isolated nanoparticles/vesicles. Supermeres were found to have a distinct size peak compared to sEVs and exomeres across different tested cell media (FIG. 1B). Supermeres were found to be smaller particles than exomeres and sEVs, as expected, where none of the interferometry or nanopore-based sensing methods generally used for sEVs or exomeres are able to work, even when used with isolated particles.

The next set of measurements involved examining and comparing the zeta potentials of the isolated supermeres, exomeres, and sEVs from the different cell media (FIG. 1C). These zeta potential measurements greatly differentiated the supermeres from the exomeres and sEVs with magnitudes of zeta potential>>R_{B}T/F, which is the intensity of thermal noise/fluctuations, compared to the zeta potentials of exomeres and sEVs that were <R_{B}T/F (FIG. 1C). Since the surface charge on these particles scales exponentially with their zeta potential, this suggested that supermeres would have a net surface charge several orders higher than sEVs or exomeres. Typically, the features with the highest differences (preferably different by multiple orders) are the best features to utilize for supermere sensing as they can produce a significantly higher signal than their sEV and exomere counterparts.

The source of the higher surface charge on supermeres was also investigated. To study this, supermeres isolated from DiFi cells, which is a human colorectal cancer cell line, were treated with different types of RNase and DNase that typically target a certain type of nucleic acid. Surprisingly, it was found that RNase H reduced the zeta potential of the supermeres to <R_{B}T/F, suggesting that the higher net surface charge of supermeres is due to RNA-DNA hybrids on the surface of supermeres (FIG. 1D). It was further found that the RNase H did not solubilize the supermeres, as the supermeres remained intact with a similar size range and response to antibodies. The presence of RNA-DNA hybrids on the surface of supermeres was further confirmed for supermeres from multiple different cell cultures as they all exhibited a reduction in zeta potential to <R_{B}T/F upon treatment with RNase H (FIG. 1E).

Next, a differential detergent solubility test was performed with supermeres demonstrating that they solubilize in detergents (e.g., Triton X-100) similar to exomeres and sEVs, despite lacking a well-defined lipid mono- or bi-layer. It is possible that the hydrophobic and hydrophilic portions on the detergent molecules were able to disrupt the bonds holding the supermeres together, similar to exomeres and sEVs (FIG. 1F). This is also a key difference from protein aggregates that generally do not solubilize in detergents or require a much higher concentration of the detergent. Moreover, Surface Plasmon Resonance (SPR) further showed that these particles were solubilized as the signal was significantly reduced after dispersion of these nanoparticles.

The protein compositions in supermeres, exomeres, and sEVs derived from different cell cultures were then examined and compared using Surface Plasmon Resonance (SPR) focusing on TGFBI, ENO1, GPC1, CEA, DPEP1, HSPA13, DDR1, ENO2 (NSE), FASN, VCP, POTEE, CD9, CD81, CD63 and EGFR. These proteins were selected based on previous work where they were found to be prominent and of particular interest in these different particles. The SPR data found that the tetraspanins CD9, CD63, and CD81 were absent on supermeres and exomeres but highly prominent on sEVs, while FASN, VCP, and POTEE were mostly present on exomeres and not supermeres or sEVs (FIG. 1G). The proteins HSPA13, DDR1, and ENO2 were found to be more specific to supermeres with very small amounts in the other fractions across different cell culture media (FIG. 1G). Moreover, TGFBI, ENO1, GPC1, CEA, and DPEP1 were present in all of the three fractions in large amounts (FIG. 1G). It was also found that HSPA13 was able to pulldown a significant portion of the isolated supermeres from the pure solution, as the SPR signal was significantly reduced compared to its isotype pulldown counterpart (FIG. 1H). This implied that supermeres are colocalized with the other protein markers.

These results also suggested that, although not exclusively tested in this study, a size exclusion chromatography method used in series with immunoaffinity purification could be able to isolate supermeres. One drawback of this approach, however, is that one would not be able to isolate supermeres if one of the shared proteins across the three fractions were used, such as TGFBI, ENO1, CEA, GPC1, and DPEP1, as they would also be present on sEVs and exomeres that are difficult to resolve using Size Exclusion Chromatography. A goal of this study was to specifically quantify these proteins on supermeres from direct biofluids without producing any false signals from sEVs and exomeres that contain these same proteins.

### Example 2

### Direct Quantification From Complex Biofluids in <60 Minutes Using the Distinct Zeta Potential of Supermeres

For the specific characterization of sEVs, two different dimensions are often utilized. For example, one dimension can be a target protein colocalized with sEV-specific tetraspanins, or a target protein colocalized on particles of a specific size range. This study specifically utilized target proteins colocalized on nanoparticles having a zeta potential magnitude>>R_{B}T/F. This approach made it very specific as it was possible to design sensors that do not produce any signal for species with zeta potential magnitudes below a thermal noise level<R_{B}T/F. For instance, if capture occurs with even one of the shared proteins such as TGFBI or ENO1 that allow for binding of sEVs, exomeres, and supermeres, the signal produced by sEVs and exomeres would be close to zero.

An Ion Exchange Membrane Sensor (IEMS) was utilized, which uses an anion exchange membrane (AEM) that only allows anions to pass through the membrane upon application of an electric field. Depending on the electric field, three different regimes are observed in voltage-current response: an Ohmic (i.e., underlimiting) regime at lower voltages, a limiting regime at intermediate voltages, and an overlimiting regime at higher voltages. Once a small voltage is applied across the AEM, it follows Ohm's law because diffusive flux of the ions to the surface is significantly higher than the transport of anions across the membrane, and this regime is known as the Ohmic/underlimiting regime. However, as the electric field is increased, the diffusive flux is not able to supply enough anions for transport across the membrane, leading to stagnation of current even with increasing voltage, and this regime is known as the limiting regime. As the electric field is increased, a part of the supplied energy facilitates the formation of electroconvective instabilities where the convective vortices are able to bring enough ions for transport across the membrane, leading to another positive current-voltage slope, and this regime is the overlimiting regime. The transition between the limiting and overlimiting regimes is highly dependent on the surface charge present on the membrane. An IEMS was therefore utilized to characterize supermeres directly from biofluids.

FIG. 2A shows a schematic of the IEMS platform that was used, and FIG. 2B shows a typical current-voltage response curve (CVC) with underlimiting, limiting, and overlimiting regimes. The voltage difference was corrected for changes in membrane size/limiting current to obtain a charge signal (femto Coulomb (fC)). First, isolated supermeres from DiFi cell culture media were used to study the system and observe the signal produced across varying concentrations of supermeres (FIG. 2C). The limit of detection (LoD) was defined as three standard deviations from the limit of blank obtained by using a sample containing no supermeres (PBS or isotype control with no supermeres), obtaining a LoD of 3×10⁶ supermeres/mL of sample (~5 fM). The IEMS sensor saturated at 5×10⁹ supermeres/mL (~10 pM), giving about a 3-log dynamic range.

Next, the different fractions of DiFi-isolated supermeres (through ultracentrifugation (UC)) were compared against direct quantification of cell media biofluid as well as the fraction obtained from SPR for the same replicate of biological media (FIG. 2D). It was observed that the signals for UC+IEMS and UC+SPR produced the same result as directly using IEMS (no isolation) with the biological media for all the proteins studied, demonstrating that it was possible to directly use the IEMS platform for quantification. Additionally, to show that the source of the signal was indeed from supermeres and not from other fractions, the DiFi cell line was isolated into four different fractions-sEVs, exomeres, supermeres, and non-vesicular (NV) fractions- and different proteins were measured for each fraction (FIG. 2F). Signal was only produced when the supermere fraction was being studied and the protein being targeted was associated with supermeres (FIG. 2F). For example, even if the targeted protein was present in high abundance in the other fractions as seen in SPR, the signal produced was zero, demonstrating that signal was only produced from the supermere fraction alone.

Additionally, RNase H-treated isolated supermeres were unable to produce any signal on the IEMS membrane sensor, while the signal remained unchanged when measured on SPR as compared to pre-treatment (FIG. 2E), highlighting the importance of the zeta potential and surface charge of supermeres in producing a signal. However, since detergents delipidate supermeres, both IEMS and SPR methods did not produce a signal if detergent-treated (e.g., Triton X) supermeres were used for measurements (FIG. 2E). These results show that it is the colocalized RNA-DNA hybrid with the targeted proteins on the same supermere which, when delipidated, is not associated with the protein even though the protein concentrations remain unchanged after delipidation (non-denaturing detergent used).

The same fractionation study shown in FIG. 2D was repeated, but for two pools of human plasma instead of cell media, showing that no signal was produced for non-supermere fractions even when the abundant protein was present in those other isolated fractions (FIG. 2G). These results show that the signal produced by the described IEMS platform is from the supermere fraction as none of the other fractions produced any signal. Further, the supermere fraction from plasma was pulled down for other protein fractions, suggesting it cannot be lipoproteins either.

### Example 3

### Supermeres Outperform Exomeres and sEVs in Diagnosing Colorectal Cancer

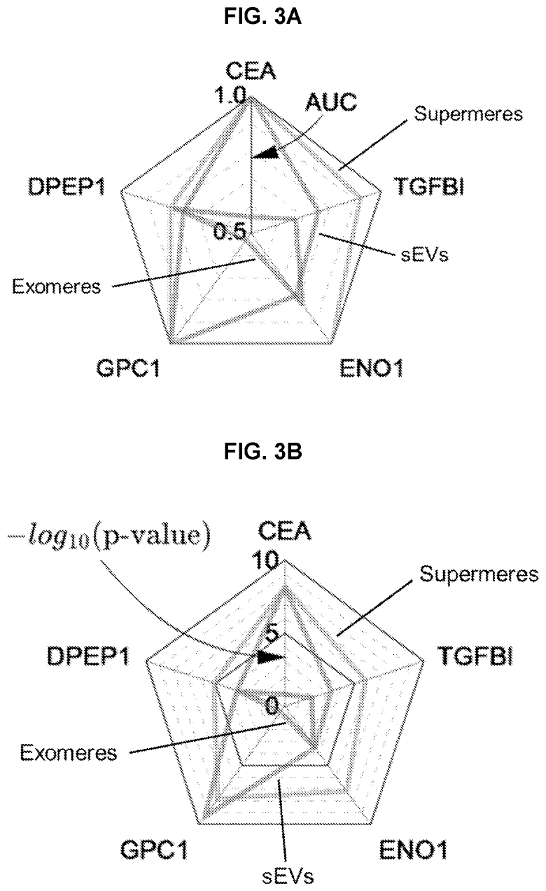

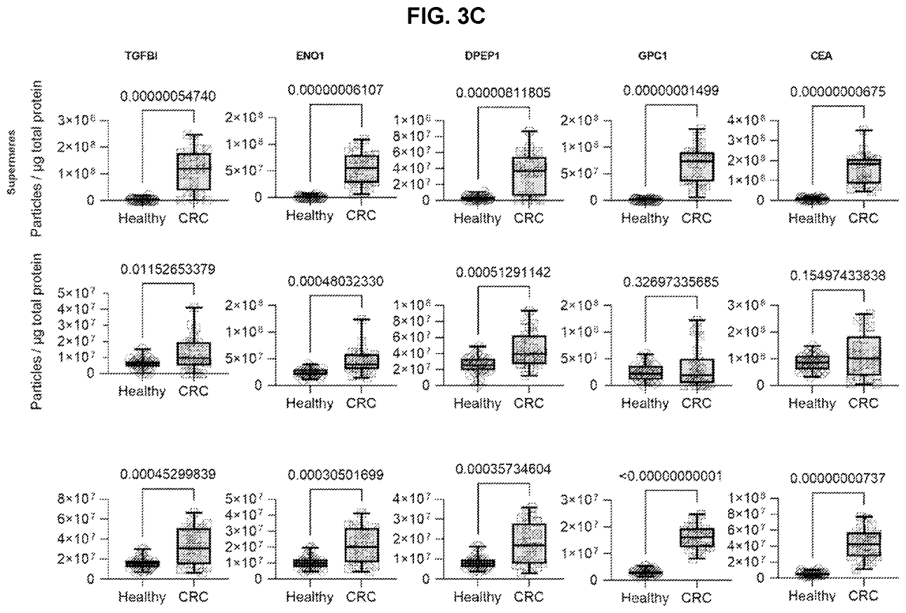

By using UC+SPR as a constant method for all three supermere, exomere, and sEV fractions, the effectiveness of these different particles in diagnosing colorectal cancer patients from healthy patients was assessed. The study included samples from 22 colorectal cancer human patients and 25 healthy human patients. The patient samples were matched for gender, race, age, ethnicity, smoking status, etc. TGFBI, CEA, ENO1, DPEP1, and GPC1 biomarker proteins were first observed, which are a group of proteins shared by all three particles (FIG. 3A-C). The spider plots shown in FIG. 3A-B show that supermeres had better AUC and p-values for these proteins as compared to sEVs and exomeres. The GPC1 protein biomarker seemed to have similar performance on both supermeres and sEVs, but overall performance was better for supermeres for all the other proteins (FIG. 3C). Additionally, exomeres did not seem to have much diagnostic capability.

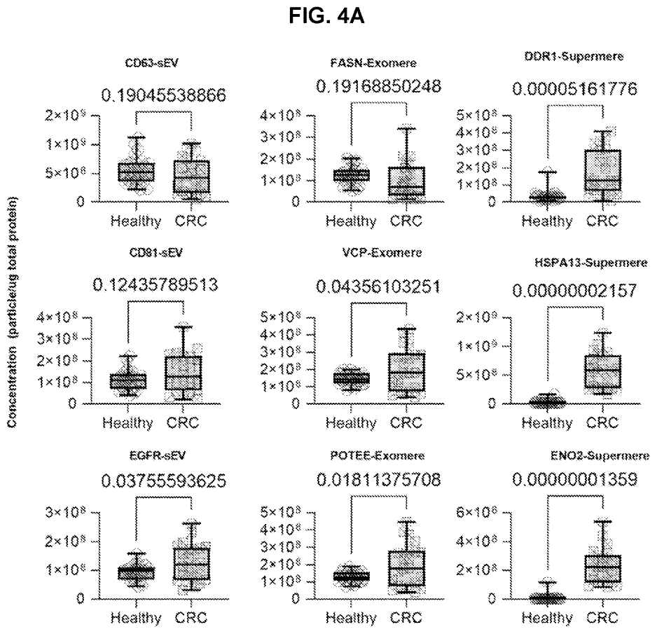

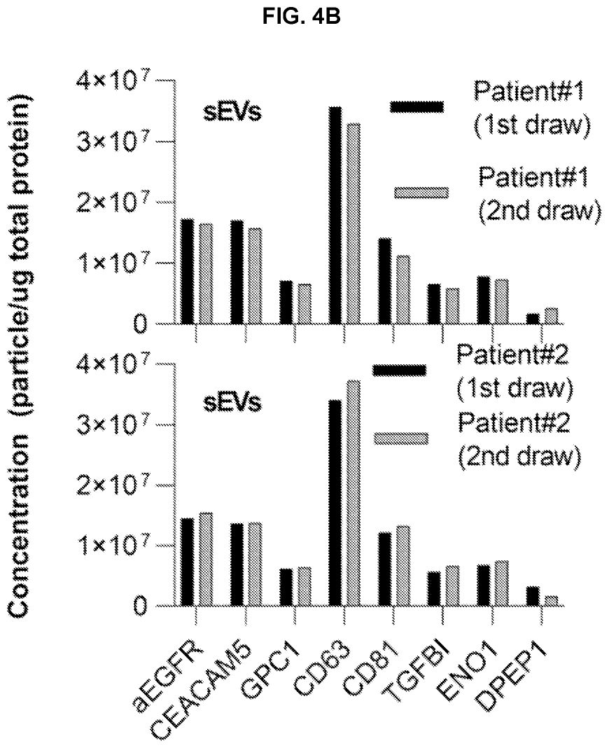

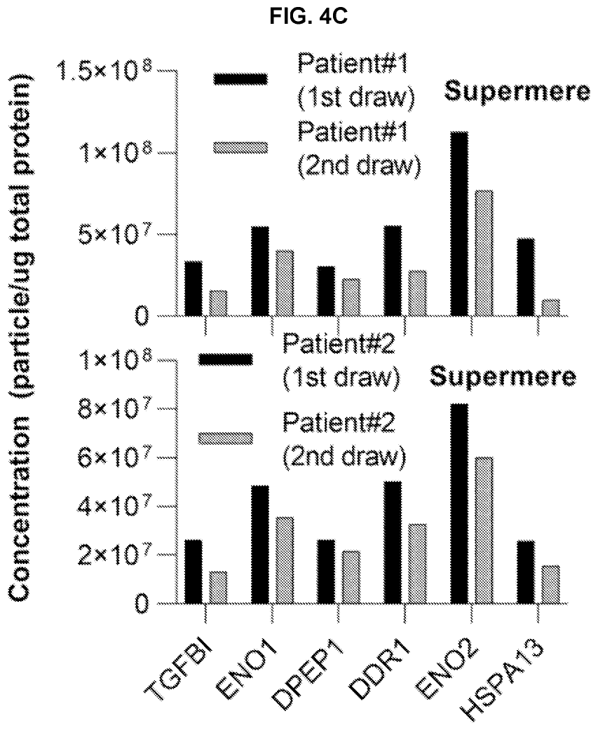

Fraction-enriched proteins were then assessed for the different fractions such as HSPA13, ENO2, and DDR1 for supermeres, VCP, POTEE, and FASN for exomeres, and CD63, CD81, CD9, and EGFR for sEVs. Since these proteins are collectively present in the vast majority of the particles, they are representative of the total count of supermeres, exomeres, and sEVs in plasma. FIG. 4A shows that supermeres were not present in the healthy patient cases that were examined, unlike exomeres and sEVs, and shows a significantly better AUC and p-values for proteins localized on supermeres in CRC patients (FIG. 4A). These results suggest that supermeres may directly be involved in the pathogenesis of cancer and are absent in healthy cases. All proteins studied on supermeres did very well at diagnosing CRC patients. Additionally, plasma samples from CRC patients who underwent resection surgery were assessed a few weeks before (1^{st} draw) and after (2^{nd} draw) surgery (FIG. 4B-C). The supermere fraction was found to be significantly reduced for the cancer patients who underwent surgery by observing the concentration difference from the 1^{st} draw (before surgery) to the 2^{nd} draw (after surgery), but there was no difference observed for the sEVs (FIG. 4B-C).

### Example 4

### IEMS Produces Similar Results as the Multi-Day UC+SPR Method

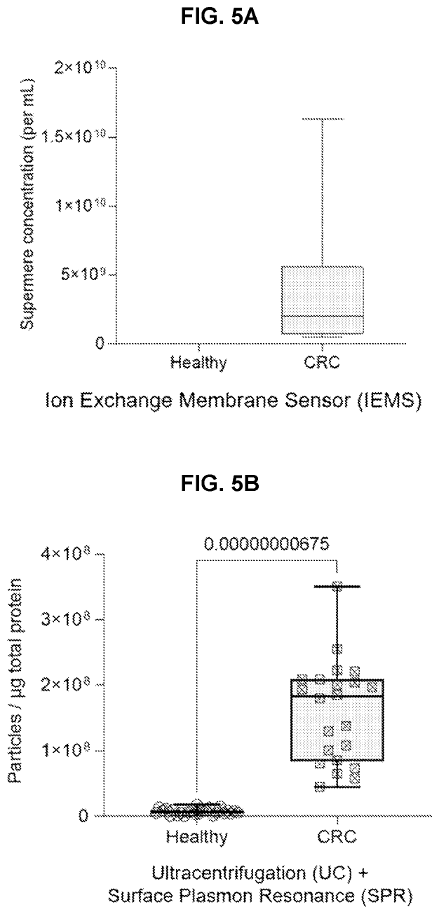

Similar assay results as the multi-day UC+SPR method were observed using the disclosed IEMS platform for the protein marker CEA in diagnosing CRC patients (FIG. 5A-B). FIG. 5A shows that a 30-minute assay using IEMS allowed for differentiation between healthy and CRC patients to be observed. FIG. 5B shows that a 4-day long UC+SPR method gave similar results as the IEMS method. IEMS was found to be much more sensitive than UC+SPR, especially in lower concentration regions where UC+SPR is not able to obtain any signal. These results demonstrated that the disclosed IEMS platform is highly suitable for characterizing supermeres directly from samples.

This study explored the different characteristics of supermeres in comparison to exomeres and sEVs, including size, zeta potential, protein distribution, and detergent solubility. Although supermeres resembled the detergent solubility of other fractions, they had a very different size distribution. However, a strikingly distinct feature of the supermeres was their highly negative zeta potential due to the presence of RNA-DNA hybrids on their surface. This property was utilized to characterize supermeres very robustly from complex media using an IEMS platform with femtomolar sensitivity. Notably, supermeres were mostly absent in fibroblast control cell culture media and healthy patients, but present in large amounts in cancer cell lines and cancer patients. Moreover, for all the proteins studied, supermeres outperformed sEVs and exomeres, highlighting the importance of studying these particles. Additionally, unlike sEVs, the supermere concentrations were reduced significantly across all tested proteins in cancer patients who underwent resection surgery.

What is claimed:

1. A method for detecting and quantifying supermeres in a sample, the method comprising:
applying the sample to a charge-sensitive ion-selective sensor that is sensitive to a high negative charge of the supermeres;
capturing the supermeres from the sample using one or more capture probes covalently attached to a surface of the charge-sensitive ion-selective sensor; and
detecting and quantifying a concentration of the supermeres in the sample using the high negative charge of the supermeres.

2. The method of claim 1, wherein the capture probes comprise an anti-DDR1 antibody, an anti-HSPA13 antibody, an anti-ENO2 antibody, or combinations thereof that bind to the supermeres.

3. The method of claim 1, wherein the sample comprises cell culture media, whole blood, serum, plasma, urine, saliva, or cerebrospinal fluid (CSF).

4. The method of claim 1, wherein the sample comprises a plasma sample from a subject.

5. The method of claim 4, wherein the subject has cancer or is at risk of developing cancer.

6. The method of claim 5, wherein the cancer comprises colorectal cancer, stomach cancer, esophageal cancer, breast cancer, lung cancer, pancreatic cancer, skin cancer, brain cancer, neck cancer, ovarian cancer, cervical cancer, prostate cancer, kidney cancer, liver cancer, blood cancer, lymphoma, or combinations thereof.

7. The method of claim 6, wherein the cancer comprises colorectal cancer.

8. The method of claim 1, wherein the sample further comprises other non-supermere extracellular particles that are not captured, detected, or quantified by the method.

9. The method of claim 8, wherein the other non-supermere extracellular particles in the sample comprise extracellular vesicles (EVs), small extracellular vesicles (sEVs), exosomes, exomeres, ectosomes, microvesicles, lipoproteins, or combinations thereof.

10. The method of claim 1, wherein the high negative charge of the supermeres is due to the presence of RNA-DNA hybrids on a surface of the supermeres.

11. The method of claim 1, wherein the supermeres have a zeta potential equal to or greater than about −30 mV.

12. The method of claim 1, wherein the charge-sensitive ion-selective sensor comprises an anion exchange membrane sensor, and wherein the capture probes are covalently attached to a surface of the anion exchange membrane sensor.

13. The method of claim 12, wherein detecting and quantifying the concentration of the supermeres in the sample comprises:
removing physically adsorbed material from the surface of the anion exchange membrane sensor following capture of supermeres;
applying an electric field to the anion exchange membrane sensor;
recording a target current-voltage (I-V) signal on the anion exchange membrane sensor, wherein the sensor produces a target I-V signal current-voltage curve (CVC) with distinct underlimiting, limiting, and overlimiting regions; and
comparing the target I-V signal CVC to a baseline I-V signal CVC, wherein when supermeres are bound to the capture probes attached to the surface of the anion exchange membrane sensor, the voltage responsible for the overlimiting region of the target I-V signal CVC is shifted as compared to an overlimiting region of the baseline I-V signal CVC, thereby confirming detection of supermeres in the sample, and wherein the concentration of the supermeres in the sample is directly correlated with a voltage shift value of the overlimiting region of the target I-V signal CVC.

14. The method of claim 1, wherein the charge-sensitive ion-selective sensor comprises an ion-sensitive field-effect transistor (ISFET) sensor, and wherein the capture probes are covalently attached to a surface of the ISFET sensor.

15. The method of claim 1, wherein the method has a limit of detection of about 1×10⁵ to about 1×10⁷ supermeres/mL in the sample with 3 to 4 orders of dynamic range.

16. The method of claim 1, wherein the supermeres in the sample are detected and quantified in less than about 30 minutes.

17. A method of detecting cancer in a subject, the method comprising:
capturing supermeres from a sample from the subject using one or more capture probes; and
detecting and quantifying a concentration of one or more supermere-related cancer biomarkers in the sample from the subject.

18. The method of claim 17, wherein the capture probes comprise an anti-DDR1 antibody, an anti-HSPA13 antibody, an anti-ENO2 antibody, or combinations thereof that bind to the supermeres.

19. The method of claim 17, wherein the supermere-related cancer biomarkers comprise TGFB1, ENO1, DPEP1, GPC1, CEA, DDR1, HSPA13, ENO2, or combinations thereof.

20. The method of claim 17, wherein the sample further comprises other non-supermere extracellular particles.

21. The method of claim 20, wherein the other non-supermere extracellular particles in the sample comprise extracellular vesicles (EVs), small extracellular vesicles (sEVs), exosomes, exomeres, ectosomes, microvesicles, lipoproteins, or combinations thereof.

22. The method of claim 17, wherein the sample from the subject comprises whole blood, serum, plasma, urine, saliva, or cerebrospinal fluid (CSF).

23. The method of claim 22, wherein the sample comprises a plasma sample from the subject.

24. The method of claim 17, wherein the subject has cancer or is at risk of developing cancer.

25. The method of claim 17, wherein the cancer comprises colorectal cancer, stomach cancer, esophageal cancer, breast cancer, lung cancer, pancreatic cancer, skin cancer, brain cancer, neck cancer, ovarian cancer, cervical cancer, prostate cancer, kidney cancer, liver cancer, blood cancer, lymphoma, or combinations thereof.
